SEQUENCE LISTING (10 sequences)

SEQ ID NO: 1 - DNA, 696 bp, Artificial sequence
  nucleotide sequence for the red fluorescent protein S3-2
gagggcggccccgccctgttccagagcgacatgaccttcaaaatcttcatcgacggcgtggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccaacggcatcagccacttcgcccaggagtgcttccccgagggcctgagcatcgaccgcaccgtgcgcttcgagaacgacggcaccatgaccagccaccacacctacgagctggacggcacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgagccacttcgacagcaagatgaccttcaacggcagccgcgccatcaagatccccggcccccacttcgtgaccaccatcaccaagcagatgaaggacaccagcgacaagcgcgaccacgtgtgccagcgcgaggtgacctacgcccacagcgtgccccgcatcaccagcgccatctga

SEQ ID NO: 2 - protein, 231 aa, Artificial sequence
  amino acid sequence for the red fluorescent protein S3-2
EGGPALFQSDMTFKIFIDGVVNGQKFTIVADGSSKFPHGDFNVHAVCETGKLPMSWKPICHLIQYGEPFFARYPNGISHFAQECFPEGLSIDRTVRFENDGTMTSHHTYELDGTCVVSRITVNCDGFQPDGPIMRDQLVDILPNETHMFPHGPNAVRQLAFIGFTTADGGLMMSHFDSKMTFNGSRAIKIPGPHFVTTITKQMKDTSDKRDHVCQREVTYAHSVPRITSAI

SEQ ID NO: 3 - DNA, 708 bp, Artificial sequence
  nucleotide sequence for the red fluorescent protein JR18IC+
gagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccaacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccgacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggat

SEQ ID NO: 4 - protein, 236 aa, Artificial sequence
  amino acid sequence for the red fluorescent protein JR18IC+
EGGPALFQSDMTFKIFIDGEVNGQKFTIVADGSSKFPHGDFNVHAVCETGKLPMSWKPICHLIQYGEPFFARYPNGISHFAQECFPEGLSIDRTVRFENDGTMTSHHTYELDDTCVVSRITVNCDGFQPDGPIMRDQLVDILPNETHMFPHGPDAVRQLAFIGFTTADGGLMMGHFDSKMTFNGSRAIEIPGPHFVTIITKQMRDTSDKRDHVCQREVAYAHSVPRITSAIGSDED

SEQ ID NO: 5 - DNA, 741 bp, Artificial sequence
  nucleotide sequence for the AceGFP permutant cpT1
atggcctccaatgtgtacatcatggccgacaaggccaggaatggcatcaaggcgaacttcaaggtccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgaccacatgatcctgttcgagttcgtgaccgcagccgccatcgcccacggcaaggatgagctgtacaagggcggtactggtggcagcatgagcaagggcgccgagctgttcaccggcatcgtgcccatcctgatcgagctgaatggcgatgtgagtggccacaagttcagcgtgagcggcgagggcgagggcgatgccacctacggcaagctgaccctgaagttcatctgcaccagcggcaagctgcctgtgccctggcccaacctggtggccaccctgagctacggcgtgcagtgcttctcacgctaccccgatcacatgaagcagcacgacttcttcaagagcgccatgcctgagggctacatccaggagcgcaccatcttcttcgaggatgacggcaactacaagacgcgcgccgaggtgaagttcgagggcgataccctggtgagtcgcatcgagctgaccggcaccgatttcaaggaggatggcaacatcctgggcaataagatggagtacggctccgccaagttc

SEQ ID NO: 6 - protein, 247 aa, Artificial sequence
  amino acid sequence for the AceGFP permutant cpT1
MASNVYIMADKARNGIKANFKVRHNIEDGSVQLADHYQQNTPIGDGPVLLPDNHYLSTQSALSKDPNEKRDHMILFEFVTAAAIAHGKDELYKGGTGGSMSKGAELFTGIVPILIELNGDVSGHKFSVSGEGEGDATYGKLTLKFICTSGKLPVPWPTLVATLSYGVQCFSRYPDHMKQHDFFKSAMPEGYIQERTIFFEDDGNYKTRAEVKFEGDTLVSRIELTGTDFKEDGNILGNKMEYGSAKF

SEQ ID NO: 7 - DNA, 675 bp, Artificial sequence
  dendQ4mono nucleotide sequence
aatctgattaaagaagatatgagggttaaggtgcatatggaagggaatgtaaacgggcatgcttttgtgattgaaggggaaggaaaaggaaagccctacgaagggacacagaccgcgaacctgacagtgaaagaaggcgcgcctctcccattttcttacgacatcttgacaacagcattgcactacggaaacagagtattcaccaaataccctgaagatataccggattatttcaagcagtcgtttcctgaaggatattcctgggaaagaaccatgacttttgaagacaagggcatttgtaccatcagaagcgacataagcttggaaggtgactgctttttccaaaacgttcgttttaaggggaccaactttcccccaaatggtccagttatgcagaagaaaactttgaagtgggaaccatccacagagaagctgcacgtgcgtgatgggttgcttgtcggtaatattaacatggctctgctgcttgaaggaggtggacattacctgtgtgacttcaaaactacttacaaagcgaagaaggttgttcagttgccagatgcccattttgtggaccatcgcattgagatcttgagtaatgacagcgattacaacaaagtgaagctgtacgagcatggggtcgctcgctattctccgttgccaagtcaggcctggtag

SEQ ID NO: 8 - protein, 224 aa, Artificial sequence
  dendQ4mono amino acid sequence
NLIKEDMRVKVHMEGNVNGHAFVIEGEGKGKPYEGTQTANLTVKEGAPLPFSYDILTTALHYGNRVFTKYPEDIPDYFKQSFPEGYSWERTMTFEDKGICTIRSDISLEGDCFFQNVRFKGTNFPPNGPVMQKKTLKWEPSTEKLHVRDGLLVGNINMALLLEGGGHYLCDFKTTYKAKKVVQLPDAHFVDHRIEILSNDSDYNKVKLYEHGVARYSPLPSQAW

SEQ ID NO: 9 - DNA, 1089 bp, hydromedusa 2 from sub-order Anthomedusae
atcattcgctgatacgaagtgaaagtagcgtttgctgaaagcttcctggaattgctcctacgtatcttgaaagttgctcctacgctccaacttgtttgttcaaaatggaaggtggtccagcattatttcaatccgatatgacattcaagatcttcatcgatggagtggtgaatgatcagaaattcacgataatcgcagatggatcgtccaaattcccccatggtgacttcaacgtgcatgctgtgtgcgaaaccgggaaactcccaatgtcatggaaacctatttgtcaccttatccaatacggggagccattctttgcaaaatatcccaatggcatcagccattttgcacaggagtgctttccagaaggattaacaattgatcgaacagtcagattcgaaaatgacggcactatgacgtctcaccacacctatgagttggacggcacctgtgtcatttccaggataaccgttaattgtgacggatttcaacctgatggaccaatcatgaaagaccagcttgttgatatcctgccaactgagacacatatgttccctcatgggtccaatgctgtcagacaattgtgctacattggcttcacgacagctgatggtggtctcatgatgtcacattttgattcgaaattgacattcaatggttcgagagcaatcaagattcctggacctcatttcgttactgtgataatcaaacagatgaaagatacaagcgacaagcgtgatcatgtgtgtcaacgtgaagtcacctacgctcactcagttccacgcatcacttctgctatctaaacatcattcttaaaaggggaacatgcacatcatacttcagtgtgagggtcagtgtgagggtctttagatgtcaatttgtcgcaggtgtcacacggcgtcgtttagatgttgaaggacgaaatgcgacaaagagattaatagagactcatatttttatgtagaatcgattcattcagcccattggtaacctttttggtattttatcatcttattattgtattggcactttgtttatattttgtatgtaatgtgtaaacaattgttgaaaatacatgtcaagaacttg

SEQ ID NO: 10 - protein, 232 aa, hydromedusa 2 from sub-order Anthomedusae
MEGGPALFQSDMTFKIFIDGVVNDQKFTIIADGSSKFPHGDFNVHAVCETGKLPMSWKPICHLIQYGEPFFAKYPNGISHFAQECFPEGLTIDRTVRFENDGTMTSHHTYELDGTCVISRITVNCDGFQPDGPIMKDQLVDILPTETHMFPHGSNAVRQLCYIGFTTADGGLMMSHFDSKLTFNGSRAIKIPGPHFVTVIIKQMKDTSDKRDHVCQREVTYAHSVPRITSAI

(19) United States
(12) Patent Application Publication
(10) Pub. No.: US 2008/0153161 A1
(43) Pub. Date: Jun. 26, 2008

(54) Genetically Encoded Photosensitizers and Methods for Using Same

(76) Inventors: Konstatin A. Lukyanov, Moscow (RU); Dmitry M. Chudakov, Moscow (RU)

Correspondence Address: PATTERSON & SHERIDAN, L.L.P., 3040 POST OAK BOULEVARD, SUITE 1500, HOUSTON, TX, 77056, US

(21) Appl. No.: 11/815,501

(22) PCT Filed: Jan. 27, 2006
(86) PCT No.: PCT/IB06/01753

§ 371 Date: Aug. 3, 2007

Related U.S. Application Data
(60) Provisional application No. 60/651,122, filed on Feb. 8, 2005.
(60) Provisional application No. 60/668,701, filed on Apr. 6, 2005.
(60) Provisional application No. 60/749,812, filed on Dec. 13, 2005.

(51) Int. Cl. C12N 5/02; C07H 21/00

(52) U.S. Cl. 435/375; 536/25.3

(57) ABSTRACT

The present invention provides genetically encoded photosensitizers developed based on fluorescent or chromo-proteins. Specific genetically encoded photosensitizers and nucleic acid molecules encoding the same are provided. Also of interest are host cells, stable cell lines and transgenic organisms comprising the above-referenced nucleic acid molecules and proteins. The subject protein and nucleic acid compositions find use in a variety of different applications and methods, particularly for disrupting biomolecules, cells or cell organelles. Finally, kits for use in such methods and applications are provided.

### BACKGROUND OF THE INVENTION

1. Field of the Invention

This invention relates generally to the field of biology, medicine and chemistry. More particularly, the invention is directed to fluorescent proteins.

2. Description of the Related Art

Photosensitizers are chromophores capable of light energy translation into reactive oxygen species (ROS) production including singlet oxygen, superoxide anion radicals, hydrogen peroxide, hydroxyl radicals, or others. As applied to living cells and tissues, ROS production can lead to target damage by necrosis or apoptosis (Almeida et al., Biochim Biophys Acta. 2004, v 20, pp. 59-86). Photosensitizers found their use in photodynamic therapy (PDT), which has been used as a clinical treatment of cancer for more than 10 years, resulting in successful techniques of cancer treatment (Brown et al., Lancet Oncol. 2004, v. 5, pp 497-508). Photosensitizers are also used for the chromophore-assisted light inactivation (CALI) technique, allowing precise inactivation of the protein of interest (Marks et al., Proc Natl Acad Sci USA. 2004, v. 6, pp 9982-9987; Surrey et al., Proc Natl Acad Sci USA. 1998, v. 14, pp 4293-4298) for functional proteomics studies. Currently known photosensitizers should be added into living systems exogeneously only. This limitation constrains the area of their application and prompts one to search for genetically encoded photosensitizers.

Green fluorescent protein from Aequorea victoria (GFP) and its homologs represent the only known group of chromogenic molecules that are fully encoded in a single gene. As used herein, the term “GFP” refers to the green fluorescent protein from Aequorea victoria, including prior art versions of GFP engineered to provide greater fluorescence or fluoresce in different colors. The sequence of wild type GFP has been disclosed in Prasher et al., Gene 111 (1992), 229-33. GFP, its mutants, and homologs have found extensive use as in vivo fluorescent labels in biomedical sciences discussed in detail by Lippincott-Schwartz and Patterson in Science, 2003; 300:87-91. GFP can be used for light-induced protein inactivation in some cases (Rajfur et al., Nat Cell Biol. 2002, v. 4, pp 286-293). However GFP was shown to be inefficient photosensitizer (Surrey et al., Proc Natl Acad Sci USA. 1998, v. 14, pp 4293-4298, Tour et al., Nat. Biotechnol. 2003 v. 21, pp 1505-1508, Rajfur et al., Nat Cell Biol. 2002, v. 4, pp 286-293), presumably because the protein shell prevents generation of ROS (Marks et al., Proc Natl Acad Sci USA. 2004, v. 6, pp 9982-9987).

### SUMMARY OF THE INVENTION

The present invention relates to a method of reactive oxygen species (ROS) production using a genetically encoded photosensitizer (GEPS). In certain embodiments said GEPS is fluorescent protein that is capable of light energy translation into ROS production. In certain embodiments, said GEPS is a red or far-red fluorescent protein, having emission maxima ranging from about 550 nm to 670 nm. In certain embodiments, said GEPS comprises Asn and Ala amino acid residues in positions corresponding to 145 and 161 positions shown in SEQ ID NO: 4. In the another embodiment, said GEPS is green, yellow, cyan or blue fluorescent protein.

In certain embodiments, the GEPS is selected from the group consisting of Cnidaria or Arthropoda derived fluorescent proteins and mutants thereof. In a preferred embodiment, said GEPS is a red fluorescent mutant of Anthomedusae anm2CP chromoprotein (PCT/RU03/00474; Shagin et. al., Mol Biol Evol. 2004, 21(5):841-850, also noted to as hm2CP). Examples of amino acid compositions for such GEPS are shown in SEQ ID NOs: 2 and 4. Anthomedusae anm2CP chromoprotein nucleic acid and amino acid sequences are shown in SEQ ID NOs: 9 and 10.

In certain embodiments, the invention is directed to GEPS that is homologous, mutant, substantially the same, or identical thereto to the protein selected from the group consisting of SEQ ID NOs: 2 or 4. In certain embodiments, the invention is directed to GEPS that comprises a Asn145/Ala161 combination in amino acid composition.

In another embodiment, the invention is directed to methods of ROS production comprising the step of GEPS expression from a nucleic acid molecule encoding a GEPS.

In certain embodiments, the nucleic acid molecules of the invention encode a fluorescent protein capable of light energy translation into ROS production. In certain embodiments, said GEPS is a red or far-red fluorescent protein, having emission maxima ranging from about 550 nm to 670 nm.

In certain embodiments, the nucleic acid molecules of the invention encode GEPS having an amino acid sequence that is selected from the group consisting of SEQ ID NOs: 2 and 4, or it homologous, mutant, substantially the same, or identical thereto. Examples of nucleic acids encoding GEPS are shown in SEQ ID NOs: 1 and 3.

In certain embodiments, the nucleic acid that is homologous, substantially the same, or identical thereto to the nucleic acid of SEQ ID NOs: 1 or 3, or the nucleic acid that differs from said nucleic acid due to the degeneracy of genetic code or hybridizes thereto, and GEPS encoded by said nucleic acid and capable of ROS production are also within the scope of the invention.

In another embodiment, the invention is directed to fusion proteins comprising GEPS of the present invention and nucleic acids encoding them.

In certain embodiments, a method of ROS generation in a cell or cell compartment is provided.

Additionally, host-cells, stable cell lines, transgenic animals and transgenic plants comprising nucleic acids capable of expressing a GEPS of the present invention are provided.

In a preferred embodiment, a method to break a chosen biological molecule(s) is provided, said method comprises contacting (coupling or drawing together) said biological molecule(s) with a GEPS, contacting the biological molecule(s) with the GEPS to photoradiation sufficient to activate the GEPS, and allowing the produced ROS to break said biological molecule(s).

In another preferred embodiment, a method and composition to disrupt a cell organelle(s) is provided, said method comprising production of a GEPS fused to a suitable subcellular localization signal in a cell, exposing the cell or cell region to photoradiation sufficient to activate the GEPS, and allowing the produced ROS to disrupt the cell organelle(s).

In yet another preferred embodiment, a method and composition to block cell proliferation and/or arrest cell growth is provided, said method comprising providing a cell or cell environment (medium, fluid, etc.) with a GEPS or a GEPS fusion protein, exposing the cell to photoradiation sufficient to activate the GEPS, and allowing the produced ROS to block cell proliferation and/or arrest cell growth.

In yet another preferred embodiment, a method and composition to kill a cell is provided. Said method comprises providing a cell with a GEPS or a GEPS fusion protein, exposing the cell to photoradiation sufficient to activate the GEPS, and allowing the produced ROS to kill the cell.

In certain embodiments, the GEPS fusion protein of the methods described above comprises a GEPS operatively linked to a specific subcellular localization signal. In another embodiment, said GEPS fusion protein comprises a GEPS operatively linked to another protein.

In certain embodiments, a GEPS or GEPS fusion protein is delivered into a cell or cell environment using any known delivery system. In another embodiment, a nucleic acid comprising a GEPS or GEPS fusion coding sequence operatively linked with suitable expression regulatory elements is delivered into a cell using any known delivery system, e.g., a viral delivery system, to synthesize a GEPS or GEPS fusion protein in a cell(s) or cell environment.

In certain embodiments, the methods described above can be used to kill cell(s), stop cell growth or to block cell proliferation in cell cultures, living tissues, or living organisms.

Also provided are compositions that comprise the GEPS of the present invention or a nucleic acid encoding the GEPS. Additionally, kits comprising nucleic acids or vectors or expression cassettes harboring said nucleic acids, or proteins of the present invention are provided.

In yet another embodiment, a method of producing a nucleic acid encoding a GEPS from a fluorescent protein incapable of ROS production is provided. Said method comprises (a) providing a nucleic acid molecule encoding a fluorescent or chromo-protein (b) nucleic acid molecule mutagenesis by means of site-directed mutagenesis, random mutagenesis, reshuffling of any type or a combination thereof (c) coupling of obtained nucleic acid molecules with suitable expression regulation sequences (d) expressing the proteins from said nucleic acid molecules and (e) selection of the proteins in which capacity to produce ROS or phototoxic capacity is increased.

### BRIEF DESCRIPTION OF THE DRAWINGS

FIG. 1 represents a diagram of survival of bacteria cells expressing different fluorescent or chromo-proteins after white light irradiation: 1.—AmCyan; 2. ZsGreen; 3—DsRed2; 4—asulCP; 5—EGFP; 6—AsRed2; 7—asuICPW94F; 8. dendGFP; 9.—gtenCP; 10.—aceGFP; 11—cpT1; 12.—dendQ4mono; 13—anm2CP; 14.—KillerRed; 15.—ZsGreenY66A.

FIG. 2 represents a graphic indicating the number of E. coli colonies expressing different fluorescent proteins, which grew without irradiation and after irradiation for the corresponding period of time.

FIG. 3 illustrates excitation (dotted line) and emission (solid line) spectra for KillerRed protein. Hatched and white rectangles show relative phototoxic effect from irradiation with blue (460-490 nm) and green (540-580 nm) light, respectively. Numbers above the rectangles represent the decrease in the number of viable cells after 10-min irradiation (folds).

FIG. 4 shows E. coli colonies expressing either EGFP or KillerRed as viewed in a fluorescent stereomicroscope. The EGFP colonies appear grey in FIG. 4 although they were actually viewed as green using a fluorescent stereomicroscope. The KillerRed protein expressing colonies appear white in FIG. 4 although they were actually viewed as red using the fluorescent stereomicroscope. A—the colonies after 8-h growth at 37° C. and overnight storage at 4° C. B—the same zone after 15-min irradiation with green light and following overnight growth at 37° C. Irradiation-induced killing of KillerRed expressing cells resulted in termination of growth of the white colonies.

FIG. 5 illustrates KillerRed CALI effect directed against beta-gal as a function of time. O-nitrophenyl (ONP) absorption after 15 min o-nitrophenyl-D galactoside (ONPG) incubation is proportional to β-gal enzymatic activity.

FIG. 6 illustrates quenching of KillerRed-mediated CALI effect in a β-gal assay by specific ROS quenchers. White and hatched columns show β-gal activity for the intact and irradiated samples, respectively.

FIG. 7 illustrates KillerRed-mediated light-induced inactivation of PLC delta-1 PH domain. (a) Schematic outline of the experimental system. (b) Dependence of EGFP-PH-KillerRed (circles) and EGFP-PH-DsRedExpress (squares) membrane to cytoplasm translocation on the irradiation time. (c, d, e) A confocal image of a cell expressing EGFP-PH-KillerRed triple fusion (EGFP green fluorescent signal) before (c), after (d) and 1.5 hour after (e) 10-s irradiation with green light. Note a considerable increase in the cytoplasmic signal. (f, g) Control experiment showing cell expressing EGFP-PH-DsRedExpress triple fusion (EGFP green fluorescent signal is shown as grey) before (f) and after (g) 1-min irradiation with green light. No change in signal distribution within the cell was observed.

FIG. 8 illustrates light-induced killing of eukaryotic cells expressing KillerRed. (A)—Mortality of 10 min-irradiated 293T cells coexpressing KillerRed and AcGFP1 in cytoplasm. First frame shows KillerRed red fluorescent signal (TRITC filter set) prior to irradiation. Further green fluorescent protein fluorescence (FITC filter set) is shown, images taken once per 15 minutes. Time zero is set immediately after irradiation with green light. (B) Mortality of 15-min irradiated B16 melanoma cells expressing mitochondrially localized KillerRed. First frame shows KillerRed red fluorescent signal (TRITC filter set) before irradiation. Further cells are shown in the white field, images taken once per 15 minutes. Time zero is set immediately after irradiation with green light.

### DETAILED DESCRIPTION

As summarized above the present invention is directed to methods of ROS production using a genetically encoded photosensitizer (GEPS), nucleic acid molecules encoding GEPS, and mutants, and variants thereof, as well as proteins and peptides encoded by these nucleic acids. Also provided are methods to generate a GEPS protein and nucleic acid from an initial fluorescent or chromo-protein, or non-colored, non-fluorescent homologues thereof, wherein the initial protein is incapable of producing ROS. Also provided are vectors and expression cassettes comprising a nucleic acid of the present invention, host-cells, stable cell lines and transgenic organisms comprising above-referenced nucleic acid molecules. The subject GEPS and nucleic acid find use in a variety of different applications and methods, particularly cell killing and CALI applications. Finally, kits for use in such methods and applications are provided.

GEPS Proteins

As used herein the term “photosensitizer” means a compound which absorbs radiation of one or more defined wavelengths and subsequently utilizes the absorbed energy to produce reactive oxygen species.

In certain embodiments, the present invention provides a genetically encoded photosensitizer (GEPS). In certain embodiments, said GEPS is a fluorescent protein that is capable of light energy translation into reactive oxygen species (ROS) production.

In certain embodiments, the subject protein is a fluorescent protein, by which is meant that it can be excited at one wavelength of light following which it will emit light at another wavelength, and the excitation spectra of the subject protein typically ranges from about 300 to 700 nm. The fluorescent characteristic of a fluorescent protein is one that arises from the interaction of two or more amino acid residues of the protein, and not from a single amino acid residue.

In certain embodiments, said GEPS is a red or far-red fluorescent protein, having an emission spectra ranging from about 550 nm to 670 nm. In still another embodiment, the GEPS is a green, yellow, cyan, or blue fluorescent protein. The subject protein generally has a maximum extinction coefficient that ranges from about 10,000 to 150,000 and usually from about 30,000 to 130,000.

In many embodiments, the subject protein has an absorbance maximum ranging from about 300 to 700, usually from about 350 to 650 and more usually from about 400 to 600 nm. The subject protein typically ranges in length from about 150 to 400 amino acids and usually is from about 200 to 350 amino acid residues, and generally has a molecular weight ranging from about 15 to 40 kDa, usually from about 17 to 35 kDa.

In another embodiment, the GEPS is a non-fluorescent colored protein of the GFP structural family. As used herein the term “chromoprotein” means a colored protein, which may be fluorescent, low or non-fluorescent. As used herein, the terms “chromoprotein” and “fluorescent protein” do not include luciferases, such as Renilla luciferase.

In certain embodiments, the protein of interest is a Cnidaria or Arthropoda derived engineered fluorescent, colored or homologue noncolored protein.

In certain embodiments, the invention is directed to GEPS selected from the group consisting of red fluorescent proteins related to the Anthomedusae anm2CP chromoprotein described in PCT/RU03/00474, provisional U.S. Patent Application Ser. No. 60/585,419 (SEQ ID NOS: 9, 10) and novel anm2CP mutants.

In certain embodiments, the GEPS of the invention comprises a Asn145/Ala161 combination in amino acid composition. The role of this combination in GEPS phototoxicity is discussed in details in the Example section.

Examples of specific GEPS include the S3-2 red fluorescent protein (PCT/RU03/00474, SEQ ID NOs: 2) and a mutant thereof, KillerRed (also noted as JR18IC+), having an amino acid sequence shown in SEQ ID NO: 4. Mutants or derivatives of these proteins capable of producing ROS are also of particular interest. As used herein the term “mutant” refers to protein disclosed in the present invention, in which one or more amino acids are added and/or substituted and/or deleted and/or inserted at the N-terminus, and/or the C-terminus, and/or within the native amino acid sequences of the proteins of the present invention.

As used herein the term “mutant” refers to nucleic acid molecule that encode a mutant protein. Moreover, the term “mutant” refers to any shorter or longer version of the protein or nucleic acid herein. As used herein the term “derivative” refers to a mutant, or an RNA-edited, or a chemically modified, or otherwise altered nucleic acid molecule, or to a mutant, or chemically modified, or otherwise altered protein.

Mutants and derivatives can be generated using standard techniques of molecular biology as described in details in the section “Nucleic acid molecules” below. Derivatives can be also generated using standard techniques that includes RNA-editing, chemical modifications, posttranslational and posttranscriptional modifications and the like. For instance, derivatives can be generated by processes such as altered phosphorylation, or glycosylation, or acetylation, or lipidation, or by different types of maturation cleavage and the like.

Proteins that are homologues, substantially the same, or identical to the GEPS of the present invention described above are also provided.

As used herein, “homologue or homology” is a term used in the art to describe the relatedness of a nucleotide or peptide sequence to another nucleotide or peptide sequence, which is determined by the degree of identity and/or similarity between said sequences compared.

By homolog is meant a protein having at least about 50%, usually at least about 60% and more usually at least about 65% amino acid sequence identity to a reference amino acid sequence (e.g. SEQ ID NOs: 2, 4) as determined using MegAlign, DNAstar clustal algorithm as described in D. G. Higgins and P. M. Sharp, “Fast and Sensitive multiple Sequence Alignments on a Microcomputer,” CABIOS, 5 pp. 151-3 (1989) (using parameters ktuple 1, gap penalty 3, window 5 and diagonals saved 5). In many embodiments, homologs of interest have much higher sequence identity e.g., 70%, 75%, 80%, 85%, 90% (e.g., 92%, 93%, 94%) or higher, e.g., 95%, 96%, 97%, 98%, 99%, 99.5%, particularly for the sequence of the amino acids that provide the functional regions of the protein.

As used herein, an amino acid sequence or a nucleotide sequence is “substantially the same” or “substantially identical” or “substantially similar” to a reference sequence if the amino acid sequence or nucleotide sequence has at least 80% sequence identity with the reference sequence over a given comparison window. In certain embodiments, substantially similar sequences have at least 85% sequence identity, at least 90% sequence identity, at least 95% sequence identity or at least 99% sequence identity or 100% sequence identity.

In certain embodiments, the proteins of interest are capable of light energy translation into reactive oxygen species (ROS) production including at least one selected from the group consisting of singlet oxygen, superoxide anion radicals, hydrogen peroxide, or hydroxyl radicals. Protein capacity to produce ROS can be estimated directly using chemical probes or physical methods (for example, using an electron-spin-resonance spectrometer). For example, singlet oxygen can be detected using Singlet Oxygen Sensor Green Reagent (Molecular Probes) which exhibits weak blue fluorescence with excitation peaks at 372 and 393 nm in the absence of singlet oxygen, while emitting a green fluorescence (excitation/emission maxima ˜504/525 nm) in the present of singlet oxygen. Alternatively, trans-1-(2′-Methoxyvinyl)pyrene: trans-1-(2′-Methoxyvinyl)pyrene can be used to detect picomole quantities of singlet oxygen in chemical and biological systems. This chemiluminescent probe does not react with other activated oxygen species such as hydroxyl radicals, superoxide or hydrogen peroxide. Hydroxyl radicals and superoxide can be detected using TEMPO-9-AC and proxyl fluorescamine probes (Molecular Probes). Each of these molecules contains a nitroxide moiety that effectively quenches its fluorescence. However, once TEMPO-9-AC or proxyl fluorescamine traps a hydroxyl radical or superoxide, its fluorescence is restored and the radical's electron spin resonance signal is destroyed, making these probes useful for detecting radicals either by fluorescence or by electron spin resonance spectroscopy. Additionally, nitro blue tetrazolium salt and other tetrazolium salts are chromogenic probes useful for superoxide determination. These probes are also widely used for detecting redox potential of cells for viability, proliferation and cytotoxicity assays.

GEPS capacity to produce ROS can be also assayed using inhibitory analysis. Specific ROS inhibitors include, for example, superoxide quenchers, e.g. superoxide dismutase in combination with catalase; superoxide anion and hydroxyl radical quencher, mannitol; and a specific quencher of singlet oxygen, sodium azide. In the presence of the specific inhibitor, the effect of certain ROS produced by GEPS is eliminated, whereas an inhibitor non-specific to the certain ROS does not alter GEPS activity. Inhibitory analysis of the GEPS of the invention in described in more detail in the Example section below.

In certain embodiments, the proteins of interest possess phototoxic capacity in response to light irradiation of a certain wavelength. Protein phototoxic capacity can be estimated by analysis of the increase of cell death rate under exposure to light irradiation of living cells (either prokaryotic or eukaryotic) containing the subject protein or in the presence of the subject protein. Protein phototoxic capacity can be also estimated by analysis of the decrease of cell proliferation rate under exposure to irradiation by a certain wavelength light of living cells (either prokaryotic or eukaryotic) containing the subject protein or in the presence of the subject protein.

In certain embodiments, the proteins of interest possess phototoxic capacity in response to light irradiation of a wavelength between about 300 nm and about 680 nm, preferably between about 450 nm and about 610 nm, more preferably between about 500 nm and about 600 nm, for example between about 520 nm and about 580 nm.

In certain embodiments, the subject proteins fold following expression in the host cell. By folding it is meant that the proteins achieve their tertiary structure that gives rise to their fluorescent quality in a short period of time. In these embodiments, the proteins fold in a period of time that generally does not exceed about 4 days, usually does not exceed about 3 days and more usually does not exceed about 1 day.

The subject proteins are isolated. As used herein the term “isolated” means a molecule or a cell that is an environment different from that in which the molecule or the cell naturally occurs. In certain embodiments, the subject proteins are substantially free of other proteins and other biological molecules, such as oligosaccharides, nucleic acids and fragments thereof, and the like, where the term “substantially free” in this instance means that less than 70%, usually less than 60% and more usually less than 50% of the composition containing the isolated protein is some other biological molecule.

In certain embodiments, the proteins are present in substantially purified form, where by “substantially purified form” means at least 95%, usually at least 97% and more usually at least 99% pure. The subject proteins may be derived from synthetic means, e.g. by expressing a recombinant nucleic acid coding sequence encoding the protein of interest in a suitable host, as described above. Any convenient protein purification procedures may be employed, where suitable protein purification methodologies are described in the Guide to Protein Purification, (Deuthser ed., Academic Press, 1990). For example, a lysate may be prepared from the original source and purified using HPLC, exclusion chromatography, gel electrophoresis, affinity chromatography, and the like.

In certain embodiments, the protein of present invention is engineered from an initial fluorescent or chromo-protein, or non-colored, non-fluorescent homologue thereof, wherein the initial protein is incapable of producing ROS or has a low capacity to produce ROS under exposure to irradiation by light of a certain wavelength. In another embodiment, the subject protein is a naturally occurring protein capable of producing ROS.

Fragments of the proteins are also provided. Biologically active fragments and/or fragments corresponding to functional domains, and the like are of a particular interest. Fragments of interest are polypeptides that are typically at least about 30 amino acids in length, usually at least about 50 amino acids in length, preferably of at least about 75 or 100 amino acids in length and may be as long as 300 amino acids in length or longer, but will usually not exceed about 250 amino acids in length, where the fragment will have a stretch of amino acids of at least about 25 amino acids that is identical to the subject protein, and usually at least about 45 amino acids, and in many embodiments at least about 50 amino acids in length. In some embodiments, the subject polypeptides are about 25 amino acids, about 50, about 75, about 100, about 125, about 150, about 200, or about 250 amino acids in length, up to the entire length of the protein. In some embodiments, a protein fragment retains all or substantially all of the specific properties of the wild type protein.

Also provided are fusion proteins comprising a protein of the present invention, or fragments thereof, fused, for example, to a degradation sequence, a sequence of subcellular localization (e.g. nuclear localization signal, peroximal targeting signal, Golgi apparatus targeting sequence, mitochondrial targeting sequence, etc.), a signal peptide, or any protein or polypeptide of interest. Fusion proteins may comprise for example, a GEPS of the present invention and a second polypeptide (“the fusion partner”) fused in-frame at the N-terminus and/or C-terminus of the GEPS. Fusion partners include, but are not limited to, polypeptides that can bind antibodies specific to the fusion partner (e.g., epitope tags), antibodies or binding fragments thereof, polypeptides that provide a catalytic function or induce a cellular response, ligands or receptors or mimetics thereof, and the like. In such fusion proteins, the fusion partner is generally not naturally associated with the fluoro/chromo-protein portion of the fusion protein, and is typically not a GEPS of subject invention or derivative/fragment thereof.

The term “operatively linked” or the like, when used to describe fusion proteins, refers to polypeptide sequences that are placed in a physical and functional relationship to each other.

The term “operatively linked” or the like, when used to describe a link with expression regulatory elements means that said regulatory elements can direct the expression of the linked DNA sequence which encodes a GEPS or GEPS fusion protein.

Also provided are antibodies that bind specifically to the GEPS of the present invention. Suitable antibodies may be produced using the techniques known in the art. For example, polyclonal antibodies may be obtained as described in (Harlow and Lane Antibodies: A Laboratory Manual, (1988) Cold Spring Harbor Laboratory Press, Cold Spring Harbor, N.Y.) and monoclonal antibodies may be obtained as described in (Goding Monoclonal Antibodies: Principles and Practice: Production and Application of Monoclonal Antibodies in Cell Biology, Biochemistry and Immunology; 3rd edition, (1996) Academic Press). Chimeric antibodies including humanized antibodies as well as single-chain antibodies and antibody fragments such as Fv, F(ab′)2 and Fab are also of interest.

Nucleic Acid Molecules

Also provided are nucleic acid molecules encoding GEPS as well as mutants of these proteins, as well as fragments thereof. A nucleic acid molecule as used herein is a DNA molecule, such as a genomic DNA molecule or a cDNA molecule, or a RNA molecule, such as a mRNA molecule. In particular, said nucleic acid molecules is a cDNA molecule having an open reading frame that encodes a GEPS of the invention or fragment thereof and is capable, under appropriate conditions, of being expressed as a GEPS or protein fragment (polypeptide) according to the invention. The invention also encompasses nucleic acids that are substantially similar to, identical to, derived from, or mimetics of the nucleic acids encoding proteins or protein fragments of the present invention. The subject nucleic acids are present in an environment other than their natural environment; e.g., they are isolated, present in enriched amounts, or are present or expressed in vitro or in a cell or organism other than their naturally occurring environment. Examples of nucleic acids encoding GEPS are shown in SEQ ID NOs: 1 and 3.

In addition, degenerate variants of the nucleic acids that encode the proteins of the present invention are also provided. Degenerate variants of nucleic acids comprise replacements of the codons of the nucleic acid with other codons encoding the same amino acids. In particular, degenerate variants of the nucleic acids are generated to increase their expression in a host cell. In this embodiment, codons of the nucleic acid that are non-preferred or a less preferred in genes in the host cell are replaced with the codons over-represented in coding sequences in genes in the host cell, wherein said replaced codons encode the same amino acid. Humanized versions of the nucleic acids of the present invention are of particular interest. As used herein, the term “humanized” refers to changes made to the nucleic acid sequence to optimize the codons for expression of the protein in mammalian (human) cells (Yang et al., Nucleic Acids Research (1996) 24: 4592-4593). See also U.S. Pat. No. 5,795,737 which describes humanization of proteins, the disclosure of which is herein incorporated by reference. Examples of degenerate variants of interest are described in more details in experimental parts, infra.

The subject nucleic acids may be isolated and obtained in substantially purified form. Substantially purified form means that the nucleic acids are at least about 50% pure, usually at least about 90% pure and are typically “recombinant”, i.e., flanked by one or more nucleotides with which it is not normally associated on a naturally-occurring chromosome in its natural host organism.

Also provided are methods to produce nucleic acid molecules encoding a GEPS from the nucleic acid molecules encoding GFP-like proteins incapable of producing ROS or having a low capacity to produce ROS under exposure to irradiation by light of a certain wavelength. Said methods comprise (a) providing a nucleic acid molecule encoding a fluorescent or chromo-protein or a non-fluorescent and non-colored homolog thereof; (b) nucleic acid molecule mutagenesis (c) coupling of obtained nucleic acid molecules with suitable expression regulation sequences (d) expressing the proteins from said nucleic acid molecules and (e) selection of the proteins in which capacity to produce ROS or phototoxic capacity is increased.

Mutants or derivatives can be generated on a template nucleic acid selected from the described-above nucleic acids by modifying, deleting or adding one or more nucleotides in the template sequence, or a combination thereof, to generate a variant of the template nucleic acid. The modifications, additions or deletions can be introduced by any method known in the art (see for example Gustin et al., Biotechniques (1993) 14: 22; Barany, Gene (1985) 37: 111-123; and Colicelli et al., Mol. Gen. Genet. (1985) 199:537-539, Sambrook et al., Molecular Cloning: A Laboratory Manual, (1989), CSH Press, pp. 15.3-15.108) including error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-directed mutagenesis, random mutagenesis, gene reassembly, gene site saturated mutagenesis (GSSM), synthetic ligation reassembly (SLR), or a combination thereof. The modifications, additions or deletions may be also introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof. In some embodiments, fluorescent proteins encoded by mutant or derived nucleic acids have the same fluorescent or biochemical properties as the initial fluorescent protein. In other embodiments, mutant or derived nucleic acids encode fluorescent proteins with altered properties.

Examples of mutations that can lead to increased protein capacity of light-induced ROS production includes introduction of an Asn145/Ala161 combination in an amino acid sequence of a fluorescent protein, wherein the positions of the amino acid residues corresponds to positions in the protein sequence shown in SEQ ID NO: 4. The key role of this combination for protein capacity of light-induced ROS production is discussed in details in the Example section below.

Protein capacity to produce ROS can be estimated directly as described in the “GEPS protein” section above. Protein phototoxic capacity can be estimated by comparisons of the vitality or proliferation rate of cells producing said protein after or without irradiation with light. Selection of the nucleic acids encoding proteins in which capacity to produce ROS or phototoxic capacity is increased can be performed by any methods known in the art. For example dead cells potentially comprising phototoxic GEPS variants can be labeled using propidium iodide dye, which cannot enter live cells and is included in immunofluorescent staining protocols to identify dead cells selected using a fluorescence-activated cell sorter, and used for amplification of GEPS of interest. In another way a GEPS can be selected using a chromophore-assisted light inactivation (CALI) technique, by measuring inactivation or destruction of the molecule attached to GEPS after light irradiation.

Also provided are nucleic acids that encode fusion proteins comprising a protein of the present invention, or fragments thereof that are discussed in more details in the “GEPS protein” section above.

Also provided are vectors or other nucleic acid constructs comprising the subject nucleic acids. Suitable vectors include viral and non-viral vectors, plasmids, cosmids, phages, etc., preferably plasmids, and used for cloning, amplifying, expressing, transferring, etc. of the nucleic acid sequence of the present invention in the appropriate host. The choice of appropriate vector is well within the skill of the art, and many such vectors are available commercially. To prepare the constructs, the partial or full-length nucleic acid is inserted into a vector typically by means of DNA ligase attachment to a cleaved restriction enzyme site in the vector. Alternatively, the desired nucleotide sequence can be inserted by homologous recombination in vivo, typically by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

Also provided are expression cassettes or systems used inter alia for the production of the subject GEPS or fusion proteins thereof or for replication of the subject nucleic acid molecules. The expression cassette may exist as an extrachromosomal element or may be integrated into the genome of the cell as a result of introduction of said expression cassette into the cell. For expression, the gene product encoded by the nucleic acid of the invention is expressed in any convenient expression system, including, for example, bacterial, yeast, insect, amphibian, or mammalian systems. In the expression vector, a subject nucleic acid is operatively linked to a regulatory sequence that can include promoters, enhancers, terminators, operators, repressors and inducers. Methods for preparing expression cassettes or systems capable of expressing the desired product are known for a person skilled in the art.

The nucleic acids of the present invention, e.g. having the sequence of SEQ ID NOs: 1, 3, the corresponding cDNAs, full-length genes and constructs can be generated synthetically by a number of different protocols known to those of skill in the art. Appropriate nucleic acid constructs are purified using standard recombinant DNA techniques as described in, for example, Sambrook et al., Molecular Cloning: A Laboratory Manual, 2^{nd} Ed., (1989) Cold Spring Harbor Press, Cold Spring Harbor, N.Y., and under regulations described in, e.g., United States Dept. of HHS, National Institute of Health (N1H) Guidelines for Recombinant DNA Research.

Cell lines which stably express the proteins of present invention can be selected by the methods known in the art (e.g. the co-transfection with a selectable marker such as dhfr, gpt, neomycin, hygromycin allows the identification and isolation of the transfected cells that contain the gene integrated into a genome).

The above-described expression systems may be used in prokaryotic or eukaryotic hosts. Host-cells such as E. coli, B. subtilis, S. cerevisiae, insect cells in combination with baculovirus vectors, or cells of a higher organism such as vertebrates, e.g., COS 7 cells, HEK 293, CHO, Xenopus oocytes, etc., may be used for production of the protein.

When any of the above-referenced host cells, or other appropriate host cells or organisms are used to replicate and/or express the nucleic acids of the invention, the resulting replicated nucleic acid, expressed protein or polypeptide is within the scope of the invention as a product of the host cell or organism. The product may be recovered by an appropriate means known in the art.

Transgenics

The nucleic acids of the present invention can be used to generate transgenic organisms or site-specific gene modifications in cell lines. Transgenic cells of the subject invention include one or more nucleic acids according to the subject invention present as a transgene. For the purposes of the invention any suitable host cell may be used including prokaryotic (e.g. Escherichia coli, Streptomyces sp., Bacillus subtilis, Lactobacillus acidophilus, etc.) or eukaryotic host cells. Transgenic organisms of the subject invention can be a prokaryotic or a eukaryotic organism including bacteria, cyanobacteria, fungi, plants and animals, in which one or more of the cells of the organism contains a heterologous nucleic acid of the subject invention introduced by way of human intervention, such as by transgenic techniques well known in the art.

The isolated nucleic acid of the present invention can be introduced into the host by methods known in the art, for example infection, transfection, transformation or transconjugation. Techniques for transferring the nucleic acid molecules (i.e. DNA) into such organisms are widely known and provided in references such as Sambrook et al. (Molecular Cloning: A Laboratory Manual, 3^{nd} Ed., (2001) Cold Spring Harbor Press, Cold Spring Harbor, N.Y.).

In one embodiment, the transgenic organism can be a prokaryotic organism. Methods on the transformation of prokaryotic hosts are well documented in the art (for example see Sambrook et al. Molecular Cloning: A Laboratory Manual, 2nd edition (1989) Cold Spring Harbor Laboratory Press and Ausubel et al., Current Protocols in Molecular Biology (1995) John Wiley & Sons, Inc).

In another embodiment, the transgenic organism can be a fungus, for example yeast. Yeast is widely used as a vehicle for heterologous gene expression (for example see Goodey et al Yeast biotechnology, D R Berry et al, eds, (1987) Allen and Unwin, London, pp 401-429) and by King et al Molecular and Cell Biology of Yeasts, E F Walton and G T Yarronton, eds, Blackie, Glasgow (1989) pp 107-133). Several types of yeast vectors are available, including integrative vectors, which require recombination with the host genome for their maintenance, and autonomously replicating plasmid vectors.

Another host organism is an animal. Transgenic animals can be obtained by transgenic techniques well known in the art and provided in references such as Pinkert, Transgenic Animal Technology: a Laboratory Handbook, 2nd edition (2203) San Diego: Academic Press; Gersenstein and Vintersten, Manipulating the Mouse Embryo: A Laboratory Manual, 3rd ed, (2002) Nagy A. (Ed), Cold Spring Harbor Laboratory; Blau et al., Laboratory Animal Medicine, 2nd Ed., (2002) Fox J. G., Anderson L. C., Loew F. M., Quimby F. W. (Eds), American Medical Association, American Psychological Association; Gene Targeting: A Practical Approach by Alexandra L. Joyner (Ed.) Oxford University Press; 2nd edition (2000). For example, transgenic animals can be obtained through homologous recombination, where the endogenous locus is altered. Alternatively, a nucleic acid construct is randomly integrated into the genome. Vectors for stable integration include plasmids, retroviruses and other animal viruses, YACs, and the like.

The nucleic acid can be introduced into the cell, directly or indirectly by introduction into a precursor of the cell, by way of deliberate genetic manipulation, such as by microinjection or by infection with a recombinant virus or with a recombinant viral vector and the like. The term genetic manipulation does not include classical cross-breeding, or in vitro fertilization, but rather is directed to the introduction of a recombinant nucleic acid molecule. This nucleic acid molecule may be integrated within a chromosome, or it may be extrachromosomally replicating DNA.

DNA constructs for homologous recombination will comprise at least a portion of a nucleic acid of the present invention, wherein the gene has the desired genetic modification(s), and includes regions of homology to the target locus. DNA constructs for random integration need not include regions of homology to mediate recombination. Conveniently, markers for positive and negative selection may be included. Methods for generating cells having targeted gene modifications through homologous recombination are known in the art. For various techniques for transfecting mammalian cells, see Keown et al., Meth. Enzymol. (1990) 185:527-537.

For embryonic stem (ES) cells, an ES cell line may be employed, or embryonic cells may be obtained freshly from a host, such as a mouse, rat, guinea pig, etc. Such cells are grown on an appropriate fibroblast-feeder layer or grown in the presence of leukemia inhibiting factor (LIF). Transformed ES or embryonic cells may be used to produce transgenic animals using the appropriate technique described in the art.

The transgenic animals may be any non-human animals including non-human mammal (e.g. mouse, rat), a bird or an amphibian, etc., and used in functional studies, drug screening and the like. Representative examples of the use of transgenic animals include those described infra.

Transgenic plants also may be produced. Methods of preparing transgenic plant cells and plants are described in U.S. Pat. Nos. 5,767,367; 5,750,870; 5,739,409; 5,689,049; 5,689,045; 5,674,731; 5,656,466; 5,633,155; 5,629,470; 5,595,896; 5,576,198; 5,538,879; 5,484,956; the disclosures of which are herein incorporated by reference. Methods of producing transgenic plants also are reviewed in Plant Biochemistry and Molecular Biology (eds. Lea and Leegood, John Wiley & Sons) (1993) pp. 275-295 and in Plant Biotechnology and Transgenic Plants (eds. Oksman-Caldentey and Barz), (2002) 719 p.

For example, embryogenic explants comprising somatic cells may be used for preparation of the transgenic host. Following cell or tissue harvesting, exogenous DNA of interest is introduced into the plant cells, where a variety of different techniques is available for such introduction. With isolated protoplasts, the opportunity arises for introduction via DNA-mediated gene transfer protocols, including incubation of the protoplasts with naked DNA, such as plasmids comprising the exogenous coding sequence of interest in the presence of polyvalent cations (for example, PEG or PLO); or electroporation of the protoplasts in the presence of naked DNA comprising the exogenous sequence of interest. Protoplasts that have successfully taken up the exogenous DNA are then selected, grown into a callus, and ultimately into a transgenic plant through contact with the appropriate amounts and ratios of stimulatory factors, such as auxins and cytokinins.

Other suitable methods for producing plants may be used such as a “gene-gun” approach or Agrobacterium-mediated transformation available to those skilled in the art.

Methods of Use

The GEPS of the present invention (as well as other components of the subject invention described above) find use in a variety of different applications. For example, they may be used in the methods for disruption of selected biological objects like biomolecules (e.g. proteins, nucleic acids), cellular structures and cells, or to block cell proliferation. Representative uses for each of these types of proteins will be described below, where the uses described herein are merely exemplary and are in no way meant to limit the use of the proteins of the present invention to those described.

In a preferred embodiment relating to the method for cell killing, the GEPS find use in photodynamic therapy applications, e.g. in a methods for the treatment of neoplastic and non-neoplastic diseases, like eye disorders, renal disorders, and skin disorders as well as age related macular degeneration. In these methods, the GEPS are localized in target tissues or cells, and subsequently activated with an appropriate wavelength of light. Light activation of the GEPS generates ROS, which are toxic to target cells and tissues.

A significant drawback of PDT is the limited penetration depth of light. Currently used clinical photosensitizers have an action spectrum peaking at about 620-690 nm. The light of this wavelength light penetrates biological tissues at a depth of only several millimeters. Thus, for clinical therapy, two photon excitation is of particular interest, as it allows for infra-red radiation to be used for excitation. Two-photon excitation-PDT provides a deeper penetration depth of light into diseased tissue because mammalian tissues have a spectral window between 800 and 1100 nm, determined by the absorption of water and endogenous constituents such as hemoglobin, melanin, etc. Potentially, two-photon excitation can be used in PDT (Goyan and Cramb, Photochem Photobiol. 72(6):821-7, 2000), as it was reported that chemical photosensitizers react similarly in one- and two-photon activation (Fisher et al., Photochem Photobiol. 66(2):141-55, 1997). GFP-like proteins have absorption maxima peaked at 400-600 nm (Labas et al., Proc Natl Acad Sci USA. 2; 99(7):4256-61, 2002). For the KillerRed protein two-photon excitation is optimal at about 1000-1100 nm, which coincides well with the maximum penetration of the biological tissues. For example, red fluorescent DsRed is effectively excited using 1000 nm two-photon excitation (Heikal et al., Proc Natl Acad Sci USA. 24; 97(22):11996-2001, 2000).

In one embodiment a composition comprising a GEPS protein, or a fragment, or a fusion thereof is delivered into target cells or tissues. In another embodiment, a nucleic acid encoding a GEPS protein, or a fragment, or a fusion thereof, operatively linked to suitable expression regulatory elements is delivered into target cells or tissues to provide GEPS production.

A GEPS protein or GEPS nucleic acid can be delivered into target cells or tissues using any suitable protein delivery systems, for example using liposomes, nanocapsules, microparticles, lipid particles, vesicles, and the like. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles. The formation and use of such systems is generally known to those of skill in the art (see for example, Lasic, Trends Biotechnol. 16 (7): 307-21, 1998; Takakura, Nippon Rinsho 56 (3): 691-95, 1998; Chandran et al., Indian J. EXP. BIOL. 35 (8): 801-09, 1997; Margalit, Crit. Rev. Ther. Drug Carrier Syst. 12 (2-3): 233-61, 1995; U.S. Pat. No. 5,567,434; U.S. Pat. No. 5,552,157; U.S. Pat. No. 5,565,213; U.S. Pat. No. 5,738,868; U.S. Pat. No. 5,795,587; Quintanar-Guerrero et AL., Drug Dev. Ind. Pharm. 24 (12): 1113-28, 1998; Couvreur et AL., Crit. Rev. THER. Drug Carrier Syst. 5 (1): 1-20, 1988; zur Muhlen et AL., Eur. J. Pharm. Biopharm. 45 (2): 149-55, 1998; Zambaux et a I., J. Controlled Release 50 (1-3): 31-40, 1998; and U.S. Pat. No. 5,145,684).

Selectivity of GEPS composition delivery can be enhanced by attaching a GEPS to molecular delivery systems that have high affinity for target tissues. For example, a monoclonal antibody directed against cancer-associated antigens can be used. The use of GEPS immunoconjugates offer improved GEPS delivery specificity and broadens the GEPS applicability for photodynamic therapy. In addition to antibodies, other delivery systems can be used to facilitate the transport of the GEPS to the appropriate area of treatment. For example, heparin as well as heparan sulfate proteoglycans can be used.

In preferred embodiments, a nucleic acid encoding a GEPS operatively linked to suitable expression regulatory elements can be delivered into target cells or tissues using bacterial or viral delivery systems. It was found that some bacterial species and viruses can preferentially accumulate and replicate in solid tumors and metastases (Fox et al., Gene Ther. 3(2): 173-8, 1996; Pawelek et al., Cancer Res. 15; 57(20):4537-44. 1997; Sznol et al., J Clin Invest. 105(8):1027-30, 2000; Yoon et al., Curr Cancer Drug Targets. 1 (2):85-107, 2001; Nemunaitis J. Oncology (Huntingt) 16(11):1483-92, 2002; Chakrabarty J. Bacteriol. 185(9):2683-6. 2003). This bacterial and viral feature has been used for anti-cancer therapy. Recent studies on nude mice harboring various tumors have shown that the real-time monitoring of the growing tumors is possible by using GFP labeled bacteria or viruses (Yu et al., Anal Bioanal Chem. 377(6):964-72, 2003; Yu et al., Nat. Biotechnol. 22(3):313-20, 2004). GEPS-coding gene can be introduced in a viral or bacterial genome and thus ensure selective GEPS accumulation along with replicating organism, Suitable viral delivery systems include adenovirus, lentivirus, herpesvirus, vaccinia virus and adeno-associated virus. Adenovirus, a double-stranded DNA virus, is currently the best studied gene transfer vector for delivery of a heterologous nucleic acid (for a review, see Becker et al., Meth. Cell Biol. 43:161-89, 1994; and Douglas and Curiel, Science & Medicine 4:44-53, 1997). Suitable bacterial delivery systems include, for example, Escherichia coli, Vibrio cholerae, Salmonella typhimurium, Listeria monocytogenes, etc.

In yet another preferred embodiment, a method to kill a cell or group of cells is provided, said method comprising providing a cell environment (medium, fluid, etc.) with a GEPS or GEPS fusion protein, exposing the cell including the cell environment to photoradiation sufficient to activate the GEPS, and allowing the produced ROS to disrupt the cell. In certain embodiments, a GEPS or GEPS fusion protein is produced in cell environment from the nucleic acid encoding the GEPS or GEPS fusion protein operatively linked with a suitable expression regulatory element using a suitable expression system. In another embodiment, GEPS or GEPS fusion protein is delivered into a cell environment using an artificial delivery system. In certain embodiments, the method to kill cell(s) leads to cell death by apoptosis. In another embodiment, the method to kill cell(s) leads to cell death by cell necrosis.

The fluorescent proteins of the present invention also find use in applications involving inactivation of infectious microorganisms, e.g. viruses and bacteria, in biological fluids, like blood. Contamination of blood supplies with infectious microorganisms such as HIV, hepatitis and other viruses and bacteria presents a serious health hazard for those who must receive transfusions of whole blood or administration of various blood components. This invention provides methods for treating a fluid to inactivate microorganisms which may be present therein. In one embodiment, the method comprises (a) contacting an inactivation-effective, substantially non-toxic amount of GEPS with said fluid (b) exposing the fluid to photoradiation sufficient to activate the GEPS and (c) allowing the produced ROS to inactivate microorganisms in said fluid.

Also of interest are methods to study the molecular basis of different biological processes, e.g. developmental studies. For example, eukaryotic plasmids expressing a GEPS under the control of CMV promoter can be microinjected into animal poles of Xenopus oocytes. Thus the whole embryos will contain a GEPS. After several hours of incubation at the room temperature, a group of cells of the embryo can be irradiated to provoke a cell killing effect. Abnormalities of further embryo development can be monitored. A GEPS can be also used under the control of specific promoters. Thus, the GEPS should allow spatially and temporally controlled killing of only definite cell type within organisms.

Also of interest are methods to stop cell proliferation and cell growth. Said methods comprise providing a cell or cell environment (medium, fluid, etc.) with a GEPS or GEPS fusion protein, exposing the cell to photoradiation sufficient to activate the GEPS, and allowing the produced ROS to block cell proliferation.

Also of interest are methods to break a chosen biological molecule(s). Method comprises contacting said biological molecule(s) with GEPS, exposing the contacting biological molecule(s) and GEPS to photoradiation sufficient to activate the GEPS, and allowing the produced ROS to break said biological molecule(s). For example, biological molecules to be disrupted can be proteins or nucleic acids. They can be coupled with a GEPS covalently or non-covalently, including by fusion protein generation, conjugation, etc, or drawn together. This method can be applied, for example, to study the function of molecules of interest through temporally and spatially controlled molecule inactivation. Definite protein disruption can be used also to induce cell death through necrosis or apoptosis.

Also of interest are methods to disrupt a cell organelle(s). Said methods comprise production of a GEPS fused to a suitable subcellular localization signal in a cell, exposing the cell or cell region to photoradiation sufficient to activate the GEPS, and allowing the produced ROS to disrupt the cell organelle(s). This method can be applied to study the effect of the organelle's disruption using spatially and temporally controlled organelle(s) disruption. Organelle disruption can be used also to induce cell death through necrosis or apoptosis.

Kits

Also provided by the present invention are kits for use in practicing one or more of the above-described applications. In preferred embodiments, kits may be used for selective cell killing. Kits typically include the protein of the invention as such, or a nucleic acid encoding the same preferably with the elements for expressing the subject proteins, for example, a construct such as a vector comprising a nucleic acid encoding the subject protein. The kit components are typically present in a suitable storage medium, such as a buffered solution, typically in a suitable container. Also present in the kits may be antibodies specific to the provided protein. In certain embodiments, the kit comprises a plurality of different vectors each encoding the subject protein, where the vectors are designed for expression in different environments and/or under different conditions, for example, constitutive expression where the vector includes a strong promoter for expression in mammalian cells or a promoterless vector with a multiple cloning site for custom insertion of a promoter and tailored expression, etc.

In addition to the above components, the subject kits will further include instructions for practicing the subject methods. These instructions may be present in the subject kits in a variety of forms, one or more of which may be present in the kit.

The following example is offered by way of illustration and not by way of limitation.

### EXAMPLES

### Example 1

Screening of different fluorescent and chromo-proteins for phototoxic capacity on prokaryotic cells.

Looking for a GFP-like protein, which could be used as an efficient photosensitizer, we screened different proteins for a phototoxic effect in E. coli including:
(1) Anemonia majano fluorescent protein AmCyan (also noted to as FP486, GenBank ID AF168421; Clontech);
(2) Zoanthus sp. fluorescent protein ZsGreen FP506 (also noted to as FP506, GenBank ID AF168422; Clontech);
(3) red fluorescent protein DsRed2 (Clontech);
(4) Anemonia sulcata GFP-like chromoprotein asulCP FP595 (also noted to as FP595, GenBank ID AF246709);
(5) asulCPW94F protein, a mutant of chromoprotein FP595 comprising W94F amino acid substitution;
(6) commercially available red fluorescent protein AsRed2 (Clontech);
(7) Dendronephthya sp. green fluorescent protein dendGFP (GenBank ID AF420591);
(8) Goniopora tenuidens GFP-like chromoprotein, gtenCP (GenBank ID AF383156);
(9) aceGFP (GenBank ID AY233272);
(10) circularly permutated aceGFP (GenBank ID AY233272) variant, cpT1 having nucleotide and amino acid sequences shown in SEQ ID NOS: 5, 6.;
(11) monomeric mutant of dendGFP protein, dendQ4mono, having nucleotide and amino acid sequences shown in SEQ ID NOS: 7, 8.;
(12) EGFP, the most popular Aequorea GFP mutant variant (GenBank ID U55763);
(13) chromoprotein am2CP(PCT/RU03/00474; SEQ ID NOS: 9, 10);
(14) red fluorescent protein KillerRed (JR18IC+)(SEQ ID NOS: 3, 4), developed on the basis of chromoprotein am2CP by random mutagenesis; and
(15) non-colored mutant of FP506 protein, ZsGreenY66A, containing mutation Y66A which eliminates the chromophore, was used as a negative control.

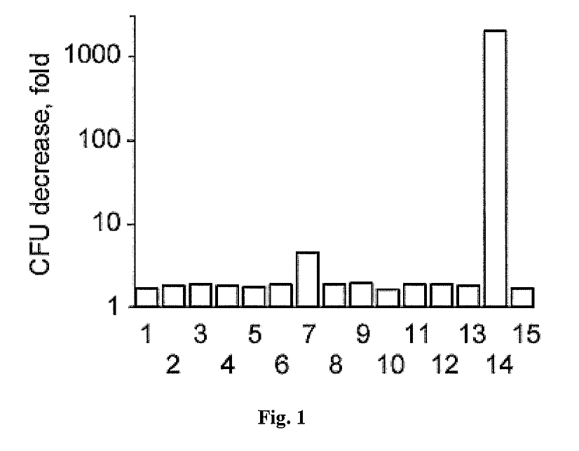

Synthetic genes encoding proteins listed above were cloned into the pQE30 vector and transformed into E. coli (XL1-Blue strain). For each protein tested, protein expressing cells were taken from one E. coli colony, diluted into 1 ml of PBS buffer and divided into two portions. One of them was irradiated with white light (light source Fiber-Light from Dolan-Jenner Industries, Inc) for 1 hour. Then both samples aliquots were sowed to the Petri dishes at different dilutions. The number of growing colonies corresponded to the number of bacteria cells surviving after irradiation (i.e. colony forming units, CFU). The CFU number for the irradiated E. coli portion was compared to non-irradiated one, thus allowing an estimate of the relative phototoxic effect for every of the proteins tested. As a result, 12 proteins tested showed neglible or zero effect, close to the negative control (non-colored protein ZsGreenY66A). One of the proteins, asulCP—W94F, showed a weak phototoxic effect, resulting in lower percent of bacteria surviving after irradiation. Another one, JR18IC+(KillerRed) showed a striking phototoxic effect, killing 99.95% of bacteria after 1 hour irradiation with white light (FIG. 1).

### Example 2

Mutational Analysis of KillerRed Phototoxic Properties.

Initially KillerRed (JR18IC+) protein was generated by random mutagenesis of wild type chromoprotein anm2CP. anm2CP was not phototoxic in E. coli as was tested in the same experiment as described in the Example 1. Comparing to the wild type chromoprotein anm2CP, KillerRed contains amino acid changes T145N and C161A (corresponding to positions 148 and 165 in Aequorea victoria GFP), which are spatially close to the chromophore and affect fluorescent properties, and a number of folding mutations. To provide for the better understanding of amino acid residues that determine phototoxic effect, a number of anm2CP mutant variants were tested as described in the Example 1, including those obtained by random mutagenesis and site-directed mutagenesis of amino acid positions 145 and 161.

Besides the Asn145/Ala161 combination, the following anm2CP 145/161 variants were generated: Gly145/Sern61, Gly145/Ala161, Ser145/Ala161, Asn145/Gly161. All these variants were shown to be non-phototoxic, indicating the key role of the Asn145/Ala161 combination. At the same time, mutants containing both Asn145 and Ala161 were phototoxic including S3-2 red fluorescent protein (PCT/RU03/00474, SEQ ID NOs: 2). Indeed, phototoxicity was most strongly expressed in KillerRed among all the variants tested. Therefore, Asn145/Ala161 combination appeared to be indispensable for the pronounced phototoxic effect in anm2CP mutants.

### Example 3

### Examination of KillerRed Phototoxic Property

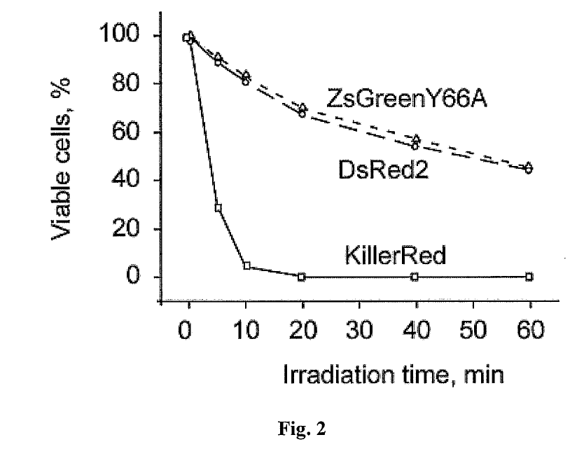

To compare the KillerRed phototoxic effect with the effect of DsRed2 fluorescent protein, pQE30 vectors encoding KillerRed, DsRed2 or ZsGreenY66A non-colored protein were prepared as described in Example 1. E. coli (XL1-Blue strain) were transformed with said pQE30 vectors. The cells expressing ZsGreenY66A was used as a negative control. We estimated the percent of cells surviving after bacteria irradiation for 5, 10, 20, 40 and 60 minutes. FIG. 2 shows the result of the experiment. The graph of FIG. 2 indicates the number of colonies, which grew without irradiation and after irradiation for the corresponding period of time. Again, red fluorescent protein DsRed2 showed almost zero effect, close to the negative control ZsGreenY66A, while KillerRed killed 96 percent of cells after 10 minutes and almost 100% of cells after 20 minutes of irradiation with white light.

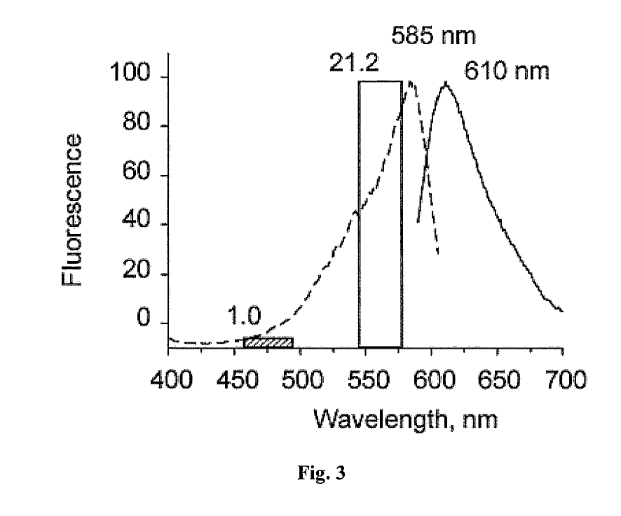

Further we roughly checked the action spectrum for KillerRed phototoxic effect (FIG. 3). A strong cell killing effect was observed upon green light irradiation (Olympus fluorescent binocular SZX12 was used as a light source, TRITC filter set) and only a weak effect upon blue light irradiation (FITC filter set), thus indicating correlation between a KillerRed absorption spectrum (peaking at 585 nm) and action spectrum for the phototoxic effect.

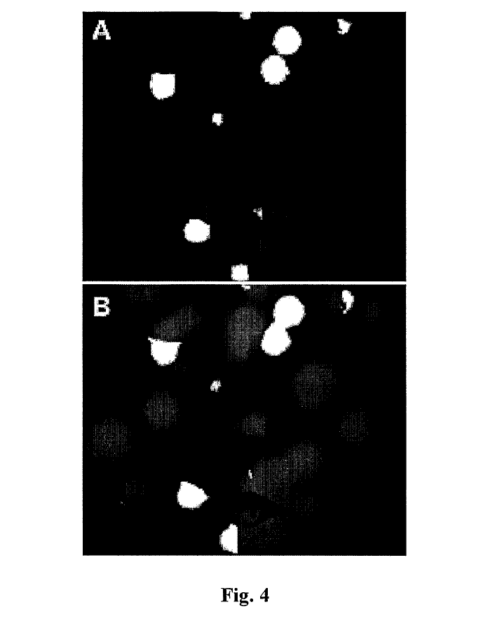

To compare phototoxic effect of KillerRed and EGFP, E. coli expressing EGFP or KillerRed prepared as described in Example 1 were mixed together and spread them onto the plate. FIG. 4A shows an area of small colonies just before intense green light illumination that results in killing of KillerRed-containing cells. FIG. 4B shows the same area upon overnight growth at 37° C. Comparing A and B photos one can clearly see that only grey (EGFP-expressing) colonies enlarged while white (KillerRed-expressing) colonies remained practically unchanged. This procedure can be generally used to perform high-throughput screening of mutant libraries to identify most effective GEPS variants.

### Example 4

### Examination of KillerRed Phototoxic Property Using CALI Technique

KillerRed ability to affect the activity of beta-galactosibase enzyme in vivo in E. coli cells was assayed. Nucleic acid encoding alpha fragment of beta-galactosidase (beta-gal) was operatively linked to the KillerRed-encoding nucleic acid and cloned into the pQE30 plasmid. The pQE30 plasmid encoding the alpha fragment of beta-gal alone was used as a control.

Both constructs were transformed into E. coli cells (XL blue strain), carrying the beta-gal gene deprived of the alpha-fragment. It is well known that translated alpha-fragment peptide is able to assemble with the rest of the enzyme, restoring its activity. Therefore when beta-gal alpha fragment is expressed as a fusion with KillerRed protein (KillerRed-alpha fragment fusion) it provides proper KillerRed targeting to the close proximity of the enzyme active center.

E. coli cells expressing beta-gal alpha-fragment alone or KillerRed-alpha fragment fusion were streaked on Petri dishes in the presence of IPTG. The resulting streaks were transferred onto Hybond-C membrane and placed on filters wetted with standard PBS media with MgSO₄ at 1 mg/ml. Streaks were illuminated with green light for 10 min using fluorescent microscope, and then membranes were transferred on the filters wetted with the PBS/MgSO₄ media containing X-Gal substrate at concentration of 1 mg/ml, and incubated at 37° C. for 30 min. Blue coloration was detected in the control streaks and non-irradiated streaks expressing KillerRed-alpha fragment fusion suggesting beta-gal activity, whereas no blue coloration was found in the irradiated area of the streaks expressing KillerRed-alpha fragment fusion. Moreover, no visible loss of beta-gal activity was found in the control E. coli cells irradiated with green light for 1 hr.

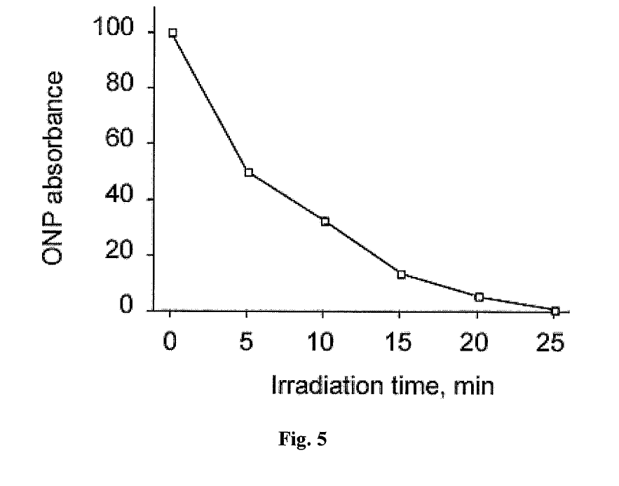

Further CALI effect directed against beta-gal as a function of time was measured (FIG. 5). XL1-Blue cells expressing KillerRed-alpha fragment fusion were ultrasonically disrupted in standard PBS buffer and 50-μl aliquots of clarified lysate were analyzed. Aliquots were either incubated under white light (1 W/cm2, light source Fiber-Light from Dolan-Jenner Industries, Inc) or kept in the dark. The entire volume of the tube was illuminated. Beta-gal activity was assayed in light-illuminated and “dark” aliquots calorimetrically. The activity of samples was measured by an orthonitrophenyl galactoside assay as described in (Horstkotte, E. et al. Photochem Photobiol., 2005, V. 81, 358-366). In the E. coli cell extract, beta-gal fused to KillerRed lost 99.4% of enzymatic activity within 25 min of white light exposure (1 W/cm2), with a half inactivation time of about 5 min. Irradiation of E. coli extracts, containing unfused beta-gal protein alone or beta-gal mixed with KillerRed had no effect on enzyme activity.

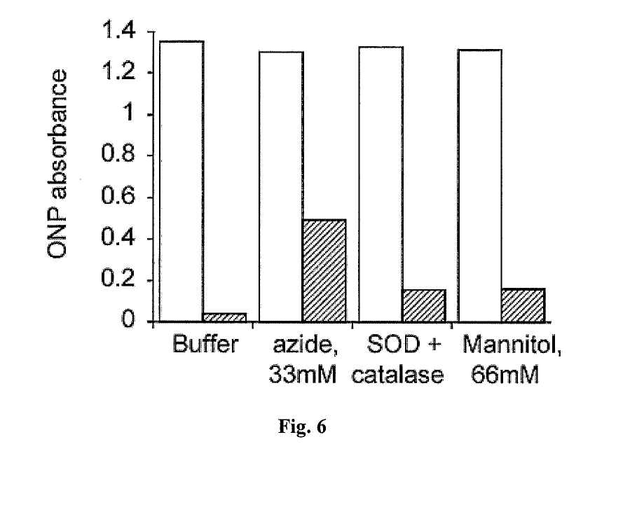

The mechanism of the KillerRed-mediated effect was tested using inhibitory analysis. A specific ROS quenchers were added to the in vitro beta-gal CALI system described above (FIG. 6). A specific superoxide quencher, superoxide dismutase (500 u/ml) in combination with catalase (500 u/ml), impeded CALI slightly. A similar effect was achieved in the presence of mannitol (67 mM), a superoxide anion or hydroxyl radical quencher. Finally, sodium azide (20 mM), a strong specific quencher of singlet oxygen, significantly reduced CALI of beta-gal. The quenching data suggest that singlet oxygen is the primary and major KillerRed-generated damaging agent.

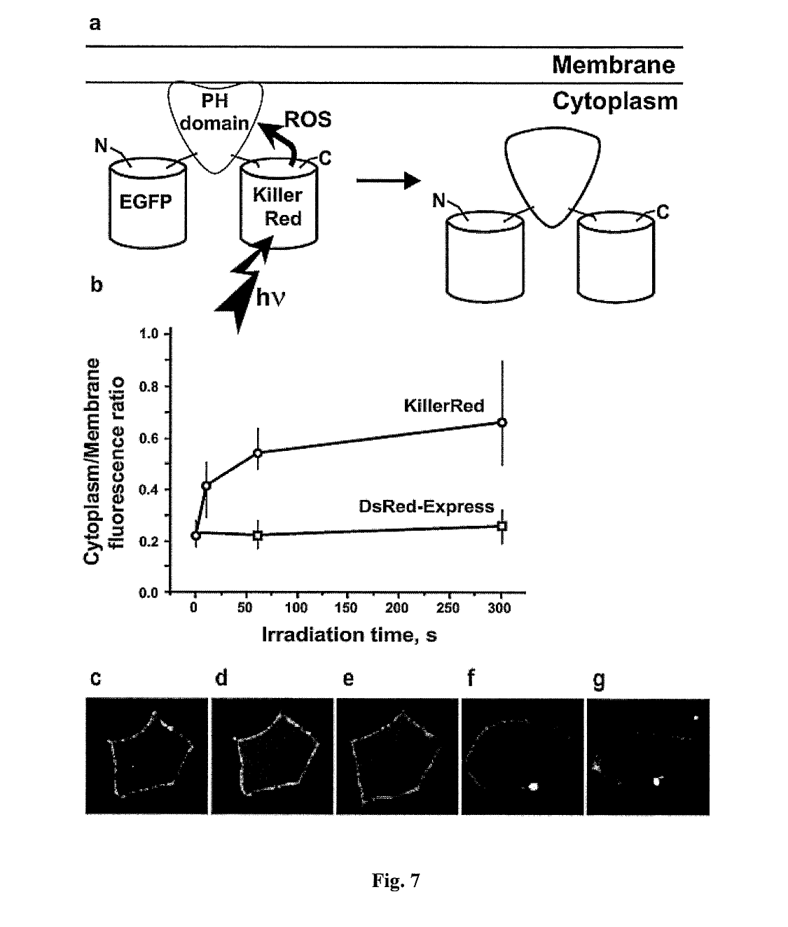

KillerRed-mediated CALI was further tested in mammalian cells (FIG. 7). The pleckstrin homology (PH) domain of phospholipase C delta-1 (PLC delta-1) was used as a model protein. The PH domain locates to the inner leaf of plasma membrane due to its high affinity for membrane phospholipids. It has been shown that in case of direct protein inactivation by dye-generated ROS, the PH domain will lose its membrane affinity and become evenly distributed throughout the cell volume.

A coding sequence for the first 224 amino acids of PLC delta-1 comprising the N-terminal PH-domain was amplified from Fetus Brain cDNA (Clontech) with specific PCR primers. The DsRed-Express gene was amplified from DsRed-Express-N1 vector (Clontech) and KillerRed was amplified from pQE30 plasmid obtained as described in the Example 1. Primers used comprised certain restriction sites that were used for following cloning of PCR products into pEGFP-Tub plasmid (Clontech) in frame with EGFP. As a result, a PH domain fusion with EGFP and triple fusions EGFP-PH-KillerRed and EGFP-PH-DsRed-Express operatively linked with CMV promoter were obtained.

The 293T cell-derived Phoenix Eco cell line was transiently transfected with these constructs. Intracellular localization of PLC delta-1 was evaluated using EGFP signal before and after CALI of the PH domain using confocal and fluorescence microscopy. Fluorescence was monitored using a Leica Confocal Microscope DM IRE2 TCS SP2. A green fluorescent signal was acquired at excitation 488 nm (laser line) and collected in the 492-538 nm wavelength range. Normally, triple fusion EGFP-PH-KillerRed is localized predominantly at the plasma membrane due to specific affinity of the PH domain to phosphatidylinositol 4,5-bisphosphate. Irradiation with intense green light leads to ROS generation by KillerRed and damage of the adjacent PH domain. As a result, the fusion protein dissociates from the membrane.

In order to initiate CALI of the PH domain, the sample was illuminated with green light (515-560 nm, mercury lamp source, x63 magnification). As expected, light-induced damage of the PH domain affected its membrane affinity dramatically. In resting cells most of the fluorescent signal located to the cell membrane, EGFP with the signal intensity ratio for cytoplasm and membrane being approximately 0.2. Upon 10-s green light irradiation (63× objective, mercury lamp, 515-560 nm filter, 7W/cm2) PH domain translocation into cytoplasm was clearly visible. If irradiated for longer time intervals, a considerable part of the PH domain translocated into cytoplasm, resulting in the essential loss of the visible membrane-cytoplasm border. The cytoplasm to membrane green fluorescent signal ratio increased to 0.5-0.9. In the negative control experiments, when a GFP—PH-DsRedExpress construct was used in place of GFP—PH-KillerRed, its cellular location did not demonstrate any dependence on green light irradiation. Even prolonged exposure of cells to green light at times and intensities sufficient to cause nearly complete bleaching of DsRed-Express fluorescence did not affect membrane tagging of the green signal. Similarly, no detectable CALI of the PH domain could be achieved in the experiments where KillerRed was expressed in the cell separately from the PH domain, in membrane or cytosol, indicating selective cis-action of KillerRed against the fused PH domain.

### Example 5

### Light-Mediated Killing of Eukaryotic Cells Expressing KillerRed

For expression in eukaryotic cells, the PCR-amplified Agel-NotI fragments from pQE30 vectors encoding KillerRed and DsRed2 (obtained as described in the Example 1) were PCR amplified and inserted in lieu of the EGFP-coding region in the pEGFP-N1 vector (Clontech).

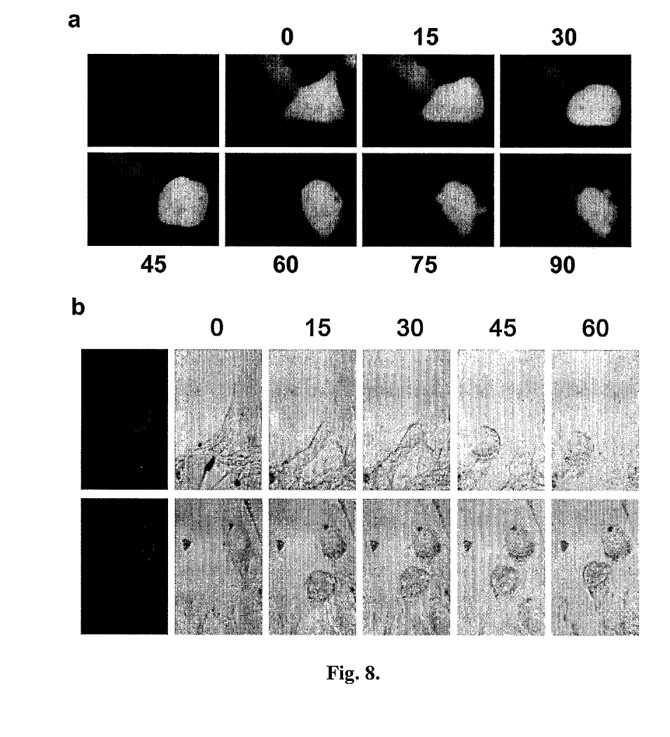

293T human kidney cells were transiently cotransfected with these vectors and the AcGFP1-C1 vector (Clontech) using Lipofectamine reagent (Invitrogen). The cells of interest were characterized with high expression levels of both green fluorescent protein AcGFP1 and KillerRed (or DsRed2 in a control experiment). Cells were irradiated with intense green light for 10 min (100× objective, 535-575 nm excitation filter, 5.8 W/cm2). Mortality of irradiated KillerRed expressing cells reached 40-60%, depending on protein concentration (FIG. 8A).

The illumination resulted in profound KillerRed photobleaching, while cell fate still could be monitored by green fluorescent protein fluorescence provided by AcGFP1. This irradiated cell started to change shape several minutes post irradiation and disintegrated within an hour and a half, indicating KillerRed phototoxicity. At the same time, neighboring cells with a lower KillerRed expression level survived irradiation, presumably due to the lower concentration of the phototoxic agent. No phototoxic effect was observed for DsRed2 expressing cells. A Leica confocal inverted microscope TCS SP2 equipped with 125 mW Ar and 1 mW HeNe lasers was used for cell imaging. A LaserCheck (Coherent) power meter was used to measure total power of the excitation light after the microscope objective.

Mitochondrial localization of a photosensitizer is known to be effective in photodynamic therapy, leading preferentially to apoptotic cell death. To provide for a more pronounced phototoxic effect, KillerRed was targeted to mitochondria by cloning it in frame with a double N-terminal mitochondrial localization signal and the resulting construct was transiently transfected into B16 melanoma cells. Exposure of mitochondrially targeted KillerRed expressing B16 melanoma cells to green light for 15 minutes using lower irradiation intensity (40× objective, 535-575 nm excitation filter, 3.3 W/cm2) led to nearly 100% cell death within 45 min after irradiation (FIG. 8B). If pre-incubated with 10 μM pancaspase inhibitor zVAD-fmk, the cells survived identical irradiation and preserved their native shape within 1.5 hour post illumination. This indicates the apoptotic way of the mitochondrially targeted KillerRed-mediated cell death.

Apart from the immediate phototoxic effect, photosensitizers can mediate postponed cellular response, such as cell growth arrest or cell death via long-term apoptotic mechanism, which can be practically more significant in terms of medical application of the photosensitizer. To monitor long-term cell survival mixed populations of cells expressing mitochondrially targeted KillerRed or EGFP were irradiated by 30-fold lower intensity green light (3.7× objective, 535-575 nm excitation filter, 115 mW/cm2) for 45 minutes. In 16 h no red fluorescent cells were observed while green cells preserved their vitality. This experiment showed that mitochondrially localized KillerRed can mediate cell death through long-term mechanisms in response to irradiation by low light intensities, potentially achievable in the photodynamic therapy.

The phototoxicity of mitochondrial KillerRed was compared with the phototoxicity of tetramethylrhodamine (TMRM)—a red fluorescent dye capable of producing ROS which accumulates in mitochondria of living cells. B16 melanoma cells were preincubated with 100 nM TMRM for 20 min at 37° C. and irradiated by 3.3 W/cm2 green light (40× objective, 535-575 nm) for time periods of 10 sec, 1 min, 5 min, and longer. While irradiation for 10 sec didn't lead to TMRM-loaded cells' death within 1.5 h interval, irradiation for 5 minutes or longer resulted in immediate cell death. Irradiation for 1 min led to cell death within 30-40 min. The effect was similar to that obtained by 15-min irradiation of the KillerRed-expressing cells under the same conditions. Basing on this experiment, we can roughly estimate that mitochondrially localized KillerRed accumulated upon cytomegalovirus (CMV IE) immediate early promoter expression demonstrates about 1/15 of TMRM phototoxic effect.

1. A method of light-induced generation of reactive oxygen species (ROS) comprising exposing a genetically encoded photosensitizer to photoradiation sufficient to activate ROS production, wherein said genetically encoded photosensitizer is a fluorescent protein capable of light-induced generation of ROS.

2. A method of claim 1, wherein said genetically encoded photosensitizer is a fluorescent protein having an emission maxima ranging from about 550 nm to 670 nm.

3. A method of claim 1, wherein the photoradiation has a wavelength between about 500 nm and about 600 nm.

4. A method of claim 1, wherein said genetically encoded photosensitizer is a fluorescent protein having an emission maxima ranging from about 550 nm to 670 nm and having Asn at position 145 and Ala at position 161, wherein positions 145 and 161 correspond to positions 145 and 161 respectively in the protein shown in SEQ ID NO: 4.

5. A method of light-induced generation of reactive oxygen species (ROS) comprising exposing a genetically encoded photosensitizer to photoradiation sufficient to activate ROS production, wherein said genetically encoded photosensitizer is a fluorescent protein capable of light-induced generation of ROS, and the genetically encoded photosensitizer is selected from the group consisting of:
(a) a protein comprising an amino acid sequence of at least 100 residues in length, either contiguous or non-contiguous, that is substantially the same as the amino acid sequence shown in SEQ ID NOs: 2 or 4;
(b) a protein having a sequence as shown in SEQ ID NO: 4; and
(c) a mutant of anm2CP chromoprotein comprising Asn at position 145 and Ala at position 161.

6. A method according to claim 1, wherein the genetically encoded photosensitizer is part of a fusion protein.

7. A method according to claim 1 wherein the reactive oxygen species (ROS) are generated in a cell or a cell compartment(s).

8. A method according to claim 7, wherein the genetically encoded photosensitizer is produced within a cell from a nucleic acid capable of expressing said genetically encoded photosensitizer.

9. A method according to claim 7, wherein the genetically encoded photosensitizer is localized within certain cell compartment(s).

10. A method according to claim 9, wherein the genetically encoded photosensitizer is localized within cell mitochondria.

11. A method according to claim 6, wherein the ROS production leads to cell death.

12. A method according to claim 6, wherein the ROS production leads to cell growth arrest.

13. A method according to claim 6, wherein the ROS production leads to stopping of cell proliferation.

14. A method according to claim 7, wherein the ROS production leads to disruption of a cell compartment.

15. A method according to claim 6, wherein the genetically encoded photosensitizer is produced within a transgenic animal from a nucleic acid capable of expressing said genetically encoded photosensitizer.

16. A method according to claim 6, wherein the genetically encoded photosensitizer is produced within a transgenic plant from a nucleic acid capable of expressing said genetically encoded photosensitizer.

17. A method according to claim 6, wherein the genetically encoded photosensitizer is produced within a stably transfected cell from a nucleic acid capable of expressing said genetically encoded photosensitizer.

18. A method according to claim 1, wherein the genetically encoded photosensitizer is in contact with a biomolecule and the ROS production leads to disruption of the biomolecule.

19. A method according to claim 18, wherein the biomolecule is linked with the genetically encoded photosensitizer.

20. A method according to claim 19, wherein the biomolecule is covalently linked with the genetically encoded photosensitizer.

21. A method according to claim 18, wherein the biomolecule is a nucleic acid molecule.

22. A method according to claim 18, wherein the biomolecule is a polypeptide.

23. A method according to claim 22, wherein said polypeptide is in a fusion protein with the genetically encoded photosensitizer.

24. A method according to claim 23, wherein said fusion protein is produced in a cell from a nucleic acid capable of expressing said fusion protein in the cell.

25. A composition providing ROS production according to claim 1, the composition comprising a genetically encoded photosensitizer, a fusion thereof, or a nucleic acid encoding a genetically encoded photosensitizer or a fusion thereof.

26. A composition providing ROS production according to claim 25, wherein the genetically encoded photosensitizer is a fluorescent protein having an emission maxima ranging from about 550 nm to 670 nm and having Asn at position 145 and Ala at position 161, wherein positions 145 and 161 correspond to positions 145 and 161 respectively in the protein shown in SEQ ID NO: 4.

27. A composition providing ROS production according to claim 1 comprising a genetically encoded photosensitizer or a nucleic acid encoding a genetically encoded photosensitizer, wherein said genetically encoded photosensitizer is selected from the group consisting of:
(a) a protein comprising an amino acid sequence of at least 100 residues in length, either contiguous or non-contiguous, that is substantially the same as the amino acid sequence shown in SEQ ID NOs: 2 or 4;
(b) a protein having a sequence as shown in SEQ ID NO: 4; and
c) a mutant of anm2CP chromoprotein comprising Asn at position 145 and Ala at position 161.

28. A method of producing a nucleic acid molecule encoding a genetically encoded photosensitizer from a nucleic acid molecule encoding a fluorescent protein incapable of ROS production, said method comprising: (a) providing a nucleic acid molecule encoding a fluorescent or chromo-protein (b) mutagenesis of the nucleic acid molecule (c) coupling of nucleic acid molecules obtained from the mutagenesis with suitable expression regulation sequences (d) expressing proteins from said nucleic acid
